SEQUENCE LISTING (17 sequences)

SEQ ID NO: 1 - DNA, 50 bp, Homo sapiens
  source 1..50: mol_type = genomic DNA
cggcgggactcccatgcgccgagggacccaagtctccactccacccacac

SEQ ID NO: 2 - DNA, 50 bp, Homo sapiens
  source 1..50: mol_type = genomic DNA
tttcccctccctggaggacgggggctccagcatattcatgatgtcaatcg

SEQ ID NO: 3 - DNA, 50 bp, Homo sapiens
  source 1..50: mol_type = genomic DNA
ttcttaaagaatgacattacagcctaacactattgtgaattaacctatcg

SEQ ID NO: 4 - DNA, 50 bp, Homo sapiens
  source 1..50: mol_type = genomic DNA
actgcgccattgttgtcttacagagaaacaaccttctaacactctcttcg

SEQ ID NO: 5 - DNA, 50 bp, Homo sapiens
  source 1..50: mol_type = genomic DNA
cgaggaatcattgagagatacttaggcaacggattttaaatgagtgaata

SEQ ID NO: 6 - DNA, 50 bp, Homo sapiens
  source 1..50: mol_type = genomic DNA
tcacttgaacaccttttattggcactaaatttgttttaggataatgaacg

SEQ ID NO: 7 - DNA, 50 bp, Homo sapiens
  source 1..50: mol_type = genomic DNA
aataattaataactatgcatgttcccagagcatcctgggacctccttccg

SEQ ID NO: 8 - DNA, 50 bp, Homo sapiens
  source 1..50: mol_type = genomic DNA
tgcagcaggggacaccagcagcgcccgaagcaggtggcggtgggagggcg

SEQ ID NO: 9 - DNA, 50 bp, Homo sapiens
  source 1..50: mol_type = genomic DNA
cgcagaggcggctgctgtccacatccacgggactcgcagatgagactcac

SEQ ID NO: 10 - DNA, 50 bp, Homo sapiens
  source 1..50: mol_type = genomic DNA
agcaaattgatgagatgcactgaaaactgcaacacctgcagcaggcatcg

SEQ ID NO: 11 - DNA, 50 bp, Homo sapiens
  source 1..50: mol_type = genomic DNA
cgactgtgggcctaataacaggtttatgtaatttaatgagcagaaatatg

SEQ ID NO: 12 - DNA, 50 bp, Homo sapiens
  source 1..50: mol_type = genomic DNA
cgccaggtaggacagacaccgactaaagtgaagatagcatggcactttgc

SEQ ID NO: 13 - DNA, 50 bp, Homo sapiens
  source 1..50: mol_type = genomic DNA
ggaattttatgtctcataaacccagccttggctgggcttggtggctcacg

SEQ ID NO: 14 - DNA, 50 bp, Homo sapiens
  source 1..50: mol_type = genomic DNA
cggaggacatagtccacgttaaataaatgacagcgactgctgtttcactg

SEQ ID NO: 15 - DNA, 50 bp, Homo sapiens
  source 1..50: mol_type = genomic DNA
atgcccttggattgcacaggcaagattaggaaattaataacatagttgcg

SEQ ID NO: 16 - DNA, 20 bp, Homo sapiens
  source 1..20: mol_type = genomic DNA
ttgggactgagcagagtgtg

SEQ ID NO: 17 - DNA, 18 bp, Homo sapiens
  source 1..18: mol_type = genomic DNA
gggagcagagtcaggaga

(19) United States
(12) Patent Application Publication
(10) Pub. No.: US 2025/0197941 A1
(43) Pub. Date: Jun. 19, 2025

(54) ANALYSIS METHOD FOR PREECLAMPSIA DIAGNOSIS USING METHYLATION LEVEL IN CPG SITE

(71) Applicant: SUNGKWANG MEDICAL FOUNDATION, Seoul (KR)

(72) Inventors: Hyun-Mee RYU, Seongnam-si, Gyeonggi-do (KR); Ji-Hyae LIM, Seongnam-si, Gyeonggi-do (KR)

(73) Assignee: SUNGKWANG MEDICAL FOUNDATION, Seoul (KR)

(21) Appl. No.: 18/683,683

(22) PCT Filed: Aug. 19, 2022
(86) PCT No.: PCT/KR2022/012378

§ 371 Date: Feb. 14, 2024

(30) Foreign Application Priority Data
Aug. 20, 2021 (KR) 10-2021-0110050

(51) Int. Cl. C12Q 1/6883

(52) U.S. Cl. CPC C12Q 1/6883; C12Q 2600/154; G01N 2800/368

(57) ABSTRACT

The present invention provides an analytical method for providing information necessary for diagnosing preeclampsia, comprising measuring a methylation level of a certain CpG site in a subject's sample.

### REFERENCE TO AN ELECTRONIC SEQUENCE LISTING

The instant application contains a Sequence Listing which has been submitted electronically in XML format and is hereby incorporated by reference in its entirety. Said XML copy, created on Jan. 31, 2025, is named SequenceListing.xml and is 16,446 bytes in size.

### TECHNICAL FIELD

The present invention relates to an analytical method for providing information necessary for diagnosing preeclampsia, using a methylation level of a certain CpG sites.

### BACKGROUND ART

Preeclampsia (PE) is a hypertensive disorder of pregnancy that affects 2˜8% of all pregnancies globally and is one of the leading causes of maternal mortality and morbidity. It is a multisystem disorder characterized by maternal new-onset hypertension and proteinuria after 20 weeks of gestation. In the absence of proteinuria, the finding of new-onset hypertension with maternal organ dysfunction, including thrombocytopenia, renal insufficiency, impaired liver function, and pulmonary edema, is enough to make the diagnosis. Therefore, the clinical phenotype varies, with the signs of the syndrome ranging from increases in blood pressure to more serious complications, including renal and liver dysfunction and seizures. Therefore, recently, it was recommended that the subclassification of PE was according to the presence or absence of severe maternal and fetal features and not according to the severity of symptoms generally known as mild, moderate, or severe. The causes and pathophysiology of PE largely remain a mystery. However, genetic, immunological, endocrine, and environmental factors have all been implicated (Espinoza J. Abnormal fetal-maternal interactions: an evolutionary value, Obstet Gynecol. 2012; 120:370-374). Numerous studies have reported alterations of gene expression in various mechanisms related to PE, including trophoblast motility and invasion, angiogenesis, cell adhesion, and immune response. Epigenetic events play a major role in these gene expression changes. This would indicate the contribution of epigenetic modifications in the various symptoms and development of PE.

Epigenetic modifications regulate gene expression without changing the DNA sequence. DNA methylation is the most common epigenetic mechanism, which primarily occurs in CpG sites and is critical for optimal placental and fetal development. A few studies investigated the role of global DNA methylation in placentas from pregnancies complicated by PE. These studies have shown that various regions of DNA in the epigenome are hyper- and/or hypo-methylated in PE placentas compared with normal placentas (Blair J D, et al., Mol Hum Reprod. 2013; 19:697-708; Ching T, et al., Mol Hum Reprod. 2014; 20:885-904; Wang T, et al. Epigenomics. 2019; 11:1003-1019; Gao W L, et al. Hypertens Res. 2011; 34:655-661; Kulkarni A, et al., DNA Cell Biol. 2011; 30:79-84; Leavey K, et al., Clin Epigenetics. 2018; 10:28). However, there is little research on DNA methylation according to severe features of PE. Therefore, comparative DNA methylation profiling analysis in PE placenta according to severe features may improve our understanding of the pathophysiology of these diseases.

### DISCLOSURE

### Technical Problem

The present inventors investigated the epigenome-wide DNA methylation patterns in the placentas of pregnant women with or without severe features of PE and normal pregnant women and identified differentially methylated CpG sites (DMCs). As the results thereof, the present inventors have found that the 15 DMCs show different patterns in DNA methylation in the PE pregnant women, independently from severe features.

Accordingly, it is an object of the present invention to provide an analytical method for providing information necessary for diagnosing preeclampsia, comprising measuring a methylation level of one or more CpG sites among the 15 CpG sites.

### Technical Solution

In accordance with an aspect of the present invention, there is provided an analytical method for providing information necessary for diagnosing preeclampsia, comprising measuring a methylation level of one or more CpG sites selected from the group consisting of cg04502985, cg14543412, cg16070018, cg24440941, cg22339775, cg08317794, cg22324103, cg24836826, cg11144986, cg04823299, cg00979438, cg20772657, cg22499381, cg12342501, and cg10288111 in a subject's sample.

In the analytical method of the present invention, the subject's sample may be a placental sample externally discharged from a pregnant woman. The measuring a methylation level may be carried out by methylation-specific quantitative real-time polymerase chain reaction (PCR).

In an embodiment, there is provided an analytical method which comprises measuring whether one or more CpG sites selected from the group consisting of cg04502985, cg14543412, cg16070018, cg24440941, cg22339775, cg08317794, cg22324103, cg24836826, cg11144986, cg04823299, cg00979438, and cg20772657 are hypermethylated.

In another embodiment, there is provided an analytical method which comprises measuring whether one or more CpG sites selected from the group consisting of cg22499381, cg12342501, and cg10288111 are hypomethylated.

### Advantageous Effects

It has been found by the present invention that methylation levels of a certain CpG sites, i.e., cg04502985, cg14543412, cg16070018, cg24440941, cg22339775, cg08317794, cg22324103, cg24836826, cg11144986, cg04823299, cg00979438, cg20772657, cg22499381, cg12342501, and cg10288111 are significantly different in preeclampsia pregnant women, compared to normal pregnant women, regardless of severe features. Therefore, the analysis method of the present invention, which comprises measuring hypermethylation or hypomethylation of the CpG sites, can be usefully applied to diagnose preeclampsia.

### DESCRIPTION OF DRAWINGS

FIG. 1 shows the hierarchical clustering of differentially methylated CpG sites (DMCs) in PE. The methylation degree values from the 850K array were applied to an independent t test (P<0.05) and a fold-change criterion (|delta average of methyl degree|≥0.2). The P values were corrected using the Benjamini and Hochberg false discovery rate method to control false positive results from multiple testing. The methylation degree values for these DMCs were subjected to hierarchical clustering. Biological samples are on the x-axis, and DMCs are on the y-axis with strong methylation indicated in the yellow and weak or absent methylation in blue. a Control versus PE. b Control versus PE with severe features. Con: control. PE: preeclampsia. PES: preeclampsia with severe features.

FIG. 2 shows the Venn diagram showing the overlap of DMCs with significant changes. NT: normal pregnant women, PT: pregnant women, PTS: PE pregnant women with severe features.

### BEST MODE FOR CARRYING OUT THE INVENTION

Preeclampsia (PE) is an obstetric disorder with significant morbidities for both the mother and fetus possibly caused by a failure of the placental trophoblast invasion. However, its pathophysiology largely remains unclear. The present inventors performed DNA methylation profiling to determine whether differential patterns of DNA methylation correlate with PE and severe features of PE. We extracted DNA from placental tissues of 13 normal, five PE, and eight PE pregnant women with severe features. Genome-wide DNA methylation analysis was performed using the Illumina HumanMethylation 850K BeadChip. The B values obtained through the Illumina HumanMethylation 850K BeadChip were subject to calibration and quantification of the Illumina array using the intensity ratio between methylated and unmethylated probes. That is, the β values were calculated by subtracting background using negative controls on the array and taking the ratio of the methylated signal intensity to the sum of both methylated and unmethylated signals. The β values of 0 to 1 were reported for each CpG site, which is related to the percentage of methylation, from 0 to 100%. Therefore, when the B value extracted from the array data for a specific CpG site in a placental sample obtained from a subject is greater than +0.2, the corresponding CpG site can be determined to be significantly hypermethylated. And. when the β value extracted from the array data for a specific CpG site in a placental sample obtained from a subject is less than-0.2, the corresponding CpG site can be determined to be significantly hypomethylated.

Significant differences were evident for 398 DMCs, including 243 DMCs in PE and 155 DMCs in PE with severe features, compared with normal placental tissues. Of these, 12 hypermethylated DMCs and three hypomethylated DMCs were observed in both PE groups, thus were independent from severe features.

As used herein, the term “CpG or CpGs” refers to a dinucleotide sequence of DNA containing cytosine and guanosine bases. These dinucleotide sequences can be methylated in human DNA. Additionally, the term “differentially methylated CpG site (DMC) or differentially methylated CpG sites (DMCs)” refers to CpG site(s) that are differentially methylated.

The present invention provides an analytical method for providing information necessary for diagnosing preeclampsia, comprising measuring a methylation level of one or more CpG sites selected from the group consisting of cg04502985, cg14543412, cg16070018, cg24440941, cg22339775, cg08317794, cg22324103, cg24836826, cg11144986, cg04823299, cg00979438, cg20772657, cg22499381, cg12342501, and cg10288111 in a subject's sample.

The sequences of the probe ID of each CpG site are SEQ ID NOs: 1 to 15, respectively. That is, SEQ ID NO: 1 is the sequence of probe ID cg04502985, SEQ ID NO: 2 is the sequence of probe ID cg14543412, SEQ ID NO: 3 is the sequence of probe ID cg16070018, SEQ ID NO: 4 is the sequence of probe ID cg24440941, SEQ ID NO: 5 is the sequence of probe ID cg22339775, SEQ ID NO: 6 is the sequence of probe ID cg08317794, SEQ ID NO: 7 is the sequence of probe ID cg22324103, SEQ ID NO: 8 is the sequence of probe ID cg24836826, SEQ ID NO: 9 is the sequence of probe ID cg11144986, SEQ ID NO: 10 is the sequence of probe ID cg04823299, SEQ ID NO: 11 is the sequence of probe ID cg00979438, SEQ ID NO: 12 is the sequence of probe ID cg20772657, SEQ ID NO: 13 is the sequence of probe ID cg22499381, SEQ ID NO: 14 is the sequence of probe ID cg12342501, and SEQ ID NO: 15 is the sequence of probe ID cg10288111.

In the analytical method of the present invention, the subject's sample refers to a sample externally discharged from the human body, including e.g., a placental sample externally discharged from a pregnant woman. The placental sample of a pregnant woman may be obtained through a biopsy conventionally performed in a maternity hospital.

The analytical method of the present invention includes measuring a methylation level of a certain CpG sites. The measuring a methylation level may be carried according to a method conventionally used in the field of biotechnology. For example, the measuring a methylation level may be carried out by methylation-specific quantitative real-time polymerase chain reaction (PCR). From the data obtained by performing the methylation-specific quantitative real-time PCR, differences in methylation level can be extracted through ΔCt. The ΔCt value is calculated as ΔCt=Ct_{MSRE}−Ctᵢₙₚᵤₜ. The smaller ΔCt value indicates higher methylation level. Therefore, the difference in methylation levels between a normal woman and a PE patient can be determined through the ΔCt value.

It has been found by the present invention that the CpG sites of cg04502985, cg14543412, cg16070018, cg24440941, cg22339775, cg08317794, cg22324103, cg24836826, cg11144986, cg04823299, cg00979438, and cg20772657 are significantly hypermethylated in preeclampsia pregnant women than in normal pregnant women, regardless of severe features. Therefore, in an embodiment, there is provided an analytical method which comprises measuring whether one or more CpG sites selected from the group consisting of cg04502985, cg14543412, cg16070018, cg24440941, cg22339775, cg08317794, cg22324103, cg24836826, cg11144986, cg04823299, cg00979438, and cg20772657 are hypermethylated. In said embodiment, if significant (e.g., Δβ>+0.2) hypermethylation is found, the subject can be classified to a patient having risk of preeclampsia.

It has been also found by the present invention that the CpG sites of cg22499381, cg12342501, and cg10288111 are significantly hypomethylated in preeclampsia pregnant women than in normal pregnant women, regardless of severe features. Therefore, in another embodiment, there is provided an analytical method which comprises measuring whether one or more CpG sites selected from the group consisting of cg22499381, cg12342501, and cg10288111 are hypomethylated. In said embodiment, if significant (e.g., Δβ<−0.2) hypomethylation is found, the subject can be classified to a patient having risk of preeclampsia.

Hereinafter, the present invention will be described more specifically by the following examples. However, the following examples are provided only for illustrations and thus the present invention is not limited to or by them.

### Examples

### 1. Material and Methods

### (1) Study Subjects

This study was approved by the Institutional Review Board (IRB) and the Ethics Committee of Cheil General Hospital (#CGH-IRB-2017-22) and Bundan CHA Hospital (CHAMC 2018-12-063-022). Singleton pregnant women who attended antenatal care at the hospital's Department of Obstetrics and Gynecology between August 2010 and August 2017 were enrolled in this study. Written informed consent was obtained from all participants before the collection of samples and subsequent analysis.

Genome-scale DNA methylation in the placenta was compared in three groups of pregnancies: PE (n=5), PE with severe features (n=8), and controls (n=13). PE was defined as hypertension (systolic blood pressure, SBP≥140 mmHg and/or diastolic blood pressure, DBP≥90 mmHg on at least two occasions 4 h apart) and proteinuria (≥300 mg in a 24 h urine collection specimen and/or ≥1+ on dipstick testing) after 20 weeks of gestation. PE was subcategorized as “PE” and “PE with severe features” according to the criteria described in Leveno K J, Spong C Y, Dashe J S, Casey B M, Hoffman B L, Cunningham F G, et al. Williams Obstetrics, 25 edition. McGraw-Hill Education; 2018. CHAPTER 40: Hypertensive disorders. The severe features of PE were defined as DBP≥110 mmHg, SBP≥160 mmHg, onset before 34 weeks of gestation, or a birth weight below the 10th percentile based on gender and gestational age at birth. Control cases were defined as women without medical and obstetric complications that presented for delivery at term (≥37 weeks of gestation). Immediately after delivery (≤30 min), placental biopsies were collected from the fetal side of the placenta. These samples (1 g) were rinsed in phosphate-buffered saline to remove any contamination with maternal blood and amniotic fluid, snap-frozen in liquid nitrogen, and stored at −80° C. until required.

### (2) Genome-Scale DNA Methylation Microarray

Genomic DNA was extracted from placental tissue with a QIAamp Tissue Kit (Qiagen, Hilden, Germany) according to the manufacturer's protocol. Placental DNA underwent sodium bisulfite conversion using an EZ DNA Methylation Kit (Zymo Research, Irvine, CA, USA). The bisulfite-converted DNA (200 ng) was hybridized to an Illumina HumanMethylationEPIC BeadChip (Illumina, Inc., San Diego, CA, USA) (850K array), which provides genome-wide coverage containing>850,000 CpG methylation sites per sample. Amplification, hybridization, washing, labeling, and scanning of the 850K array were performed by Macrogen (Seoul, Korea).

The raw data were extracted as β values for each CpG for each sample with the R watermelon package. The β values were calculated by subtracting background using negative controls on the array and taking the ratio of the methylated signal intensity to the sum of both methylated and unmethylated signals. The β values of 0 to 1 were reported for each CpG site, which is related to the percentage of methylation, from 0 to 100%. As a quality control step for Illumina array data analysis, we eliminated probes with a detection P value>0.05 in Student's t test in any sample. Probes that mapped to the sex chromosomes and/or known single nucleotide polymorphisms were removed from the analysis.

The DMCs between groups were identified based on the average DNA methylation level difference (delta beta, Δβ) comparison and significance analysis. The final set of candidate genes was constituted by false discovery rates (FDR)≤0.05, |ΔB|>0.2 (indicating>20% difference in DNA methylation), and a P value<0.05 by Student's/test.

### (3) Methylation-Specific Quantitative Real-Time PCR of DMCs

We confirmed the methylation level of the 850K array by real-time PCR using methylation specific restriction enzyme (MSRE). Samples that had been used for the 850K array were used for methylation-specific quantitative real-time PCR. The sequences of PCR primers used and POR conditions (performed with 20 μl in total) are presented in Tables 1 and 2 below.

TABLE 1

| Target | SEQ. ID. | | | Tm | Amplification |
|---|---|---|---|---|---|
| DMCs | NO. | Primer | Sequence | (° C.) | (bp) |
| cg14543412 | 16 | Forward | TTG GGA CTG AGC AGA GTG TG | 59.55 | 201 |
| cg16070018 | 17 | Reverse | GGG AGC AGA GTC AGG AGA | 60.61 | |
| cg24440941 | | | | | |
| cg22339775 | | | | | |

TABLE 2

| Target | | Tm | | | | |
|---|---|---|---|---|---|---|
| DMCs | Step | (° C.) | Time | Cycles | Component | Amount |
| cg14543412 | Pre-denaturation | 95 | 30 sec | — | TB Green Premix Ex Taq II | 10 [symbol] |
| cg16070018 | Denaturation | 95 | 5 sec | 40 cycles | Forward/Reverse primers | 10 pmol |
| cg24440941 | | | | | | each |
| cg22339775 | Annealing/ | 58 | 35 sec | | ROX Reference Dye I | 0.4 [symbol] |
| | Extension | | | | | |
| | Melting* | 75-95 | | — | Template | ~10 ng |

*Increment 0.2° C., for 10 sec

For the analysis of the DMC methylation levels, the delta (Δ) threshold cycle (Ct) value was calculated as ΔCt=Ct_{MSRE}−Ctᵢₙₚᵤₜ. The smaller the ΔCt value, the higher the methylation level of a target gene.

### (4) Statistical Analysis

Descriptive data are presented as means with standard deviation and categorical variables as proportions and counts. The methylation levels in the study groups were compared using Kruskal-Wallis tests, followed by the post hoc Bonferroni correction test for multiple comparisons, and the Mann-Whitney U test for comparisons between the two groups. Values of P<0.05 were considered statistically significant. Statistical analyses were performed with the Statistical Package for the Social Sciences version 25.0 (SPSS Inc. Chicago, IL, USA). The statistical power of this study was calculated using post hoc analysis of the G*Power program 3.1.9.2 (Heinrich-Heine-Universität, Dusseldorf, Germany). Based on an effective size of 0.8, the sample size used in our study had >90% power at an a error of 0.05 with two-tails.

### 2. Results

### (1) Clinical Characteristics of the Study Groups

The clinical characteristics of the study groups are summarized in Table 3. Maternal age, prepregnancy body mass index, gravidity, and blood pressures measured in the first trimester were not different among the three groups. However, the percentage of nulliparous and the highest blood pressures in pregnancy were increased in the two subgroups with PE, compared with the control. Proteinuria was detected in only the PE groups. There were no differences between the two subgroups of PE regarding the highest blood pressures, proteinuria, platelet count, and levels of serum creatinine. Increased levels of liver transaminases were observed in two PE pregnant women with severe features. In pregnancy outcome, the gestational age at delivery was lower in the PE with severe features, compared with the control. Birth weights and fetal growth percentage were also lower in the PE with severe features than in the others. Therefore, the percentage of neonatal intensive care unit admission was higher in the PE group with severe features than in the other groups. The gender ratio of the fetuses was not different among all the study groups.

TABLE 3

| | | | PE with |
|---|---|---|---|
| | | | severe |
| | Control | PE | feature |
| Characteristics | (n = 13) | (n = 5) | (n = 8) |
| Maternal age (years) | 35.7 ± 4.2 | 37.4 ± 5.0 | 35.6 ± 2.1 |
| Prepregnancy body mass | 24.1 ± 3.5 | 23.7 ± 4.3 | 24.8 ± 4.4 |
| index (kg/m²) | | | |
| Gravidity (n) | 2.3 ± 0.9 | 1.8 ± 0.8 | 1.9 ± 0.6 |
| Nulliparous (%) | 46.2 (6/13) | 60 (3/5) | 87.5 (7/8) |
| Blood pressure and | | | |
| proteinuria | | | |
| SBP at booking (mm Hg) * | 111.4 ± 10.9 | 127.2 ± 10.9 | 123.9 ± 14.1 |
| DBP at booking (mm Hg) ** | 64.9 ± 12.1 | 78.4 ± 4.2 | 74.8 ± 8.7 |
| Highest SBP (mm Hg) * | 122.5 ± 8.7 | 151.2 ± 7.2 | 155.0 ± 6.7 |
| Highest DBP (mm Hg) ** | 72.2 ± 7.4 | 93.4 ± 6.6 | 97.3 ± 5.9 |
| Proteinuria (dipstick result) | 0 | 2.4 ± 0.9 | 1.9 ± 0.4 |
| Pregnancy outcome | | | |
| Gestational age at delivery | 38.8 ± 1.2 | 38.4 ± 1.9 | 35.8 ± 2.4 |
| (weeks) | | | |
| Delivery <37 weeks (%) | 0 (0/13) | 0 (0/5) | 62.5 (5/8) |
| Birthweight, (kg) | 3.3 ± 0.3 | 3.1 ± 0.5 | 2.0 ± 0.5 |
| Fetal growth percentage | 46.7 ± 6.3 | 42.4 ± 7.4 | 5.4 ± 4.1 |
| Neonatal intensive care unit | 0 (0/13) | 40 (2/5) | 87.5 (7/8) |
| admission (%) | | | |
| Male infant (%) | 46.2 (6/13) | 20 (1/5) | 62.5 (5/8) |

* SBP: Systolic blood pressure
** DBP: Diastolic blood pressure

### (2) Alteration of DNA Methylation in the Whole Genome of the PE Placenta

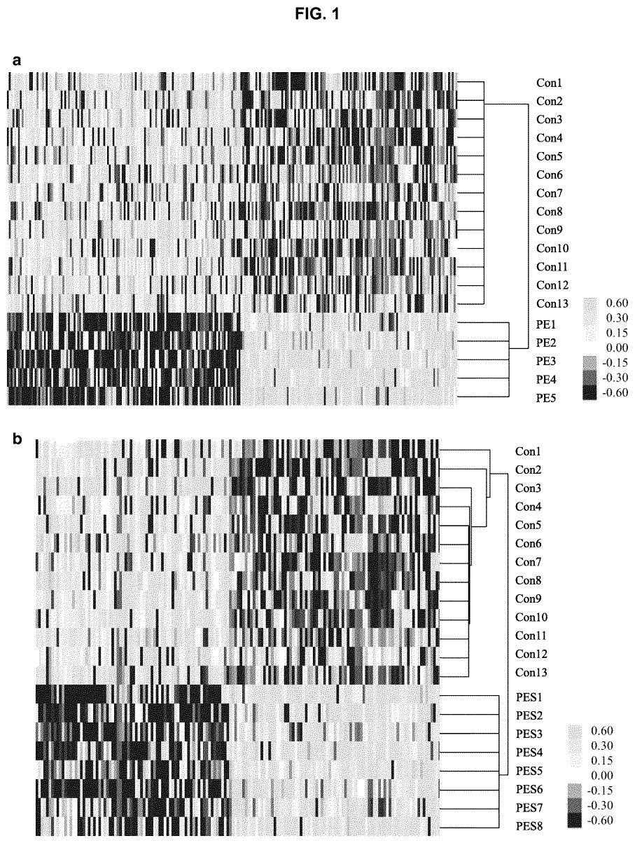

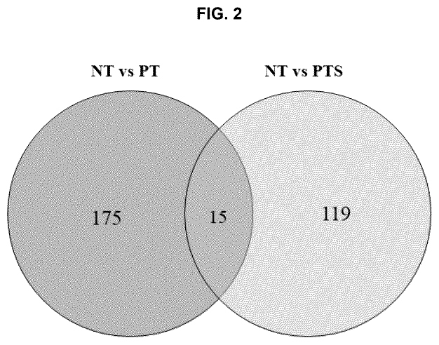

The methylation profiles of placentas were compared separately in three comparison groups: PE versus controls, PE with severe features versus controls, and PE versus PE with severe features. A total of 398 DMCs were identified: 243 in PE (51.9% hypo DMCs, n=126, and 48.1% hyper DMCs, n=117) and 155 in PE with severe features (47.7% hypo DMCs, n=74, and 52.3% hypo DMCs, n=81), as compared with controls (FIG. 1). Among them, 12 hypermethylated DMCs and three hypomethylated DMCs were commonly observed in PE, regardless of the presence or absence of severe features (FIG. 2). Table 4 shows the results of microarray analysis on the CpG sites (i.e., probe IDs) of hypermethylated DMCs showing significant differences (Δβ>0.2) in PE, compared with the control. Table 5 shows the results of microarray analysis on the CpG sites (i.e., probe IDs) of hypomethylated DMCs showing significant differences (Δβ<−0.2) in PE, compared with the control. Table 6 shows the results of microarray analysis on the CpG sites (i.e., probe IDs) of hypermethylated DMCs showing significant differences (Δβ>0.2) in PE with severe features, compared with the control. Table 7 shows the results of microarray analysis on the CpG sites (i.e., probe IDs) of hypomethylated DMCs showing significant differences (Δβ<−0.2) in PE with severe features, compared with the control. That is, the 12 hypermethylated DMCs are cg04502985, cg14543412, cg16070018, cg24440941, cg22339775, cg08317794, cg22324103, cg24836826, cg11144986, cg04823299, cg00979438, and cg20772657 (Table 4 and Table 6). And, the 3 hypomethylated DMCs are cg22499381, cg12342501, and cg10288111 (Table 5 and Table 7).

TABLE 4
DMCs showing common methylation differences in PE regardless of severe features,
among the hypermethylated DMCs in PE (compared with the control)

| | | | Subject | | | | |
|---|---|---|---|---|---|---|---|
| No | Probe ID | P value | Control | PE | Δβ > 0.2 | CHR* | MAPINFO |
| 1 | cg04502985 | 0.039 | 0.273 | 0.497 | 0.224 | 5 | 77268452 |
| 2 | cg14543412 | 0.016 | 0.108 | 0.310 | 0.202 | 6 | 26224100 |
| 3 | cg16070018 | 0.006 | 0.173 | 0.409 | 0.236 | 6 | 26224392 |
| 4 | cg24440941 | 0.029 | 0.175 | 0.379 | 0.204 | 6 | 26224668 |
| 5 | cg22339775 | 0.022 | 0.256 | 0.468 | 0.212 | 6 | 26224925 |
| 6 | cg08317794 | 0.017 | 0.575 | 0.811 | 0.236 | 11 | 131925126 |
| 7 | cg22324103 | 0.006 | 0.667 | 0.884 | 0.217 | 11 | 133175639 |
| 8 | cg24836826 | 0.031 | 0.648 | 0.946 | 0.298 | 12 | 124929963 |
| 9 | cg11144986 | 0.001 | 0.612 | 0.871 | 0.260 | 12 | 132935765 |
| 10 | cg04823299 | 0.000 | 0.541 | 0.905 | 0.364 | 14 | 24098569 |
| 11 | cg00979438 | 0.014 | 0.452 | 0.881 | 0.429 | 16 | 73199845 |
| 12 | cg20772657 | 0.005 | 0.618 | 0.826 | 0.208 | 18 | 73638846 |

*CHR: chromosome number

TABLE 5
DMCs showing common methylation differences in PE regardless of severe features,
among the hypomethylated DMCs in PE (compared with the control)

| | | | Subject | | | | |
|---|---|---|---|---|---|---|---|
| No | Probe ID | P value | Control | PE | Δβ < −0.2 | CHR* | MAPINFO |
| 1 | cg22499381 | 0.000 | 0.876 | 0.635 | −0.242 | 1 | 11024967 |
| 2 | cg12342501 | 0.025 | 0.766 | 0.404 | −0.363 | 2 | 8530521 |
| 3 | cg10288111 | 0.007 | 0.859 | 0.566 | −0.293 | 7 | 112062682 |

*CHR: chromosome number

TABLE 6
DMCs showing common methylation differences in PE regardless
of severe features, among the hypermethylated DMCs in
PE with severe features (compared with the control)

| | | | Subject | | | | |
|---|---|---|---|---|---|---|---|
| | | | | PE with | | | |
| | | | | severe | | | |
| No | Probe ID | P value | Control | features | Δβ > 0.2 | CHR* | MAPINFO |
| 1 | cg04502985 | 0.014 | 0.273 | 0.502 | 0.229 | 5 | 77268452 |
| 2 | cg14543412 | 0.003 | 0.108 | 0.361 | 0.253 | 6 | 26224100 |
| 3 | cg16070018 | 0.001 | 0.173 | 0.434 | 0.261 | 6 | 26224392 |
| 4 | cg24440941 | 0.003 | 0.175 | 0.456 | 0.281 | 6 | 26224668 |
| 5 | cg22339775 | 0.001 | 0.256 | 0.554 | 0.298 | 6 | 26224925 |
| 6 | cg08317794 | 0.001 | 0.575 | 0.826 | 0.251 | 11 | 131925126 |
| 7 | cg22324103 | 0.001 | 0.667 | 0.876 | 0.210 | 11 | 133175639 |
| 8 | cg24836826 | 0.034 | 0.648 | 0.893 | 0.245 | 12 | 124929963 |
| 9 | cg11144986 | 0.000 | 0.612 | 0.855 | 0.243 | 12 | 132935765 |
| 10 | cg04823299 | 0.000 | 0.541 | 0.753 | 0.212 | 14 | 24098569 |
| 11 | cg00979438 | 0.036 | 0.452 | 0.750 | 0.298 | 16 | 73199845 |
| 12 | cg20772657 | 0.001 | 0.618 | 0.818 | 0.200 | 18 | 73638846 |

*CHR: chromosome number

TABLE 7
DMCs showing common methylation differences in PE regardless
of severe features, among the hypomethylated DMCs in
PE with severe features (compared with the control)

| | | | Subject | | | | |
|---|---|---|---|---|---|---|---|
| | | | | PE with | | | |
| | | | | severe | | | |
| No | Probe ID | P value | Control | features | Δβ < −0.2 | CHR* | MAPINFO |
| 1 | cg22499381 | 0.005 | 0.876 | 0.575 | −0.301 | 1 | 11024967 |
| 2 | cg12342501 | 0.037 | 0.766 | 0.527 | −0.239 | 2 | 8530521 |
| 3 | cg10288111 | 0.005 | 0.859 | 0.625 | −0.234 | 7 | 112062682 |

*CHR: chromosome number

### (3) Validation of Microarray Analysis by Methylation-Specific Quantitative Real-Time PCR

To verify the microarray results of DMCs, we selected DNA regions, including two or more consecutive DMCs and MSRE recognition site. Of these, the hypermethylated DMC regions of HIST1H3E in PE, regardless of the disease's severe features (cg14543412, cg16070018, cg24440941, cg22339775) were finally selected and then confirmed their DNA methylation patterns using methylation-specific quantitative real-time PCR. The ΔCt value of HIST1H3E was significantly lower in both PE subgroups than controls. This indicates that the gene was significantly hypermethylated in both PE and PE with severe features, compared with those in controls (Table 8).

TABLE 8

| | | | | | PE with | | | | |
|---|---|---|---|---|---|---|---|---|---|
| | Target | | | | severe | | Bonferroni | | |
| Characteristics of DMCs | gene | Method | Control | PE | feature | P* | p^{a} | p^{b} | p^{c} |
| DMCs commonly changed in PE, regardless | HIST1H3E | Methyl degree of array | 0.201 | 0.418 | 0.481 | 0.001 | 0.002 | 0.007 | 0.590 |
| of severe features | | ΔCt of MSRE-qPCR | 4.895 | 3.638 | 2.485 | <0.001 | 0.025 | <0.001 | 0.056 |

### 3. Discussion

In this study, the present inventors performed DNA methylation profiling in the placentas of normal, PE, and PE pregnant women with severe features, through the microarray analysis. We found the DMCs showing different patterns in DNA methylation, independently from severe features. The methylation degree thereof was confirmed by methylation-specific real-time PCR and the results were consistent with those of the microarray analysis. Among them, the hypermethylated DMCs of HIST1H3E (cg14543412, cg16070018, cg24440941, cg22339775) appeared to have an important potential in PE. It encodes a replication-dependent histone that is a member of the histone H3 family. Transcripts contain a palindromic termination element with methylation sites. Among its related pathways, activated PKN1 stimulates the transcription of androgen receptor-regulated genes KLK2 and KLK3 and cytokine signaling in the immune system. However, the precise mechanism by which HIST1H3E is regulated by its interaction with PE has yet to be determined. In this study, the present inventors found that the TSS1500 region of HIST1H3E is hypermethylated in PE regardless of the presence or absence of severe features. This epigenetic change of HIST1H3E in PE may provide additional insight into the pathophysiology of PE generated by its misregulation.

1. An analytical method for providing information necessary for diagnosing preeclampsia, comprising measuring a methylation level of one or more CpG sites selected from the group consisting of cg04502985, cg14543412, cg16070018, cg24440941, cg22339775, cg08317794, cg22324103, cg24836826, cg11144986, cg04823299, cg00979438, cg20772657, cg22499381, cg12342501, and cg10288111 in a subject's sample.

2. The analytical method according to claim 1, wherein the subject's sample is a placental sample externally discharged from a pregnant woman.

3. The analytical method according to claim 1, wherein the measuring a methylation level is carried out by methylation-specific quantitative real-time polymerase chain reaction.

4. The analytical method according to claim 1, which comprises measuring whether one or more CpG sites selected from the group consisting of cg04502985, cg14543412, cg16070018, cg24440941, cg22339775, cg08317794, cg22324103, cg24836826, cg11144986, cg04823299, cg00979438, and cg20772657 are hypermethylated.

5. The analytical method according to claim 1, which comprises measuring whether one or more CpG sites selected from the group consisting of cg22499381, cg12342501, and cg10288111 are hypomethylated.

6. The analytical method according to claim 2, wherein the measuring a methylation level is carried out by methylation-specific quantitative real-time polymerase chain reaction.

7. The analytical method according to claim 2, which comprises measuring whether one or more CpG sites selected from the group consisting of cg04502985, cg14543412, cg16070018, cg24440941, cg22339775, cg08317794, cg22324103, cg24836826, cg11144986, cg04823299, cg00979438, and cg20772657 are hypermethylated.

8. The analytical method according to claim 2, which comprises measuring whether one or more CpG sites selected from the group consisting of cg22499381, cg12342501, and cg10288111 are hypomethylated.
